(19) United States
(12) Patent Application Publication
(10) Pub. No.: US 2005/0038266 A1
(43) Pub. Date: Feb. 17, 2005

(54) Method for producing glycerol carbonate

(76) Inventors: David Herault, Bourron-Marlotte (FR); Bernd Boutty, Meerbusch (DE); Lars Zander, Duesseldorf (DE); Albert Strube, Neuss (DE)

Correspondence Address: COGNIS CORPORATION;PATENT DEPARTMENT, 300 BROOKSIDE AVENUE, AMBLER, PA, 19002, US

(21) Appl. No.: 10/488,651

(22) PCT Filed: Aug. 29, 2002
(86) PCT No.: PCT/EP02/09631

(30) Foreign Application Priority Data
Sep. 7, 2001 (DE) 101 43 973.3

(51) Int. Cl. C07D 317/08; C07C 69/96

(52) U.S. Cl. 549/229; 558/276

(57) ABSTRACT

A process for making a purified glycerol carbonate involving: (a) providing a mixture of crude glycerol carbonate, wherein said mixture contains glycerol carbonate, glycerol and oligomeric glycerol carbonate; (b) providing a substantially water-free base component; (c) reacting the mixture with the base component to form a reaction mixture; and (d) filtering the reaction mixture to form the purified glycerol carbonate.

This invention relates to a process for the production of high-purity glycerol carbonate.

In the production of glycerol carbonate (systematically 4-hydroxymethyl-1,3-dioxolan-2-one), oligomeric products, preferably diglycerol carbonate, are always formed as secondary products in addition to the desired main product. In addition, part of the unreacted glycerol remains in the reaction product. Depending on the intended application of the glycerol carbonate, these secondary products are troublesome and may have to be removed from the crude product by complicated purification processes, such as high-vacuum distillation or chromatographic processes.

There is no known process which gives glycerol carbonate in virtually 100% pure form.

Accordingly, there is a need to provide a simple and inexpensive process that would enable glycerol carbonates to be produced in high-purity form. It has now been found that the purity of the end product can be increased by an aftertreatment.

Accordingly, the present invention relates to a process for the production of high-purity glycerol carbonate which contains glycerol and oligomeric glycerol carbonate in quantities of, in all, at most 5% by weight, characterized in that the crude glycerol carbonate is reacted in the presence of a water-free base and then worked up.

The process according to the invention starts out from crude glycerol carbonate which can be obtained by any production processes known to the expert. For example, EP 955 298 describes a process for the production of glycerol carbonates by direct reaction of glycerol in supercritical CO₂ in the presence of zeolites and ion exchange resins. EP 739 888 describes a process for the production of glycerol carbonate in which glycerol is reacted with ethylene or propylene carbonates in the presence of suitable catalysts, such as zeolites. EP 582 201 also describes a process for the production of glycerol carbonate in which glycerol is reacted with CO and oxygen at elevated temperature in the presence of transition metal catalysts. A particularly preferred embodiment of this known process uses crude glycerol carbonate obtained in accordance with the teaching of JP 95094450.

In the process according to the present invention, crude glycerol carbonate which generally contains 3 to 10% by weight of the oligomeric glycerol carbonates and around 3 to 10% by weight of free glycerol is reacted with suitable water-free bases. In the course of this reaction, the crude glycerol present or the oligomeric glycerol carbonate is substantially reduced. It is essential that the bases used are strong bases, i.e. have a pK_{B} value of at most 5, and at the same time are water-free, i.e. contain no water or at most 2 to 5% by weight water, based on the quantity of base.

A particularly preferred embodiment of the present invention is characterized by the use of bases selected from the group of tertiary amines, amines fixed to solid phases, alkali metal and alkaline earth metal silicates, preferably sodium silicates, hydroxides of the alkali metals and alkaline earth metals, carbonates and hydrogen carbonates of sodium, potassium, lithium and calcium, solid salts of sodium, potassium, lithium and calcium of alkyl alcohols. The use of sodium metasilicates, calcium hydroxide or triethylamine is particularly preferred.

The sodium silicates are the sodium salts of the various silicic acids. A distinction is made between sodium silicates where the SiO₂:Na₂O ratio is ≧2 or ≦1. The second of these two groups includes sodium metasilicate (Na₂SiO₃), MW 122.06, which is preferably used in the process according to the invention. However, it is essential to select the water-free sodium metasilicate (Mp. 1089° C.) and not the hydrated forms. Bases which themselves show little or no solubility in glycerol carbonate are particularly suitable because the end product can then be worked up simply by filtration.

In the process according to the invention, the bases are used in quantities of 0.5 to 12% by weight, preferably 1 to 10% by weight and more particularly 2 to 5% by weight, based on the crude glycerol carbonate. The desired reaction starts satisfactorily even at room temperature. However, the temperature itself is not a critical factor; the process according to the invention may also be carried out at higher or lower temperatures. However, it is preferably carried out at room temperature, i.e. at 21° C.

At 21° C., the reaction is generally over after 30 to 60 mins. Thereafter, it has proved to be of advantage first to filter the reaction mixture and then to adjust it to a pH of 4 to 5 with suitable strong acids, preferably phosphoric acid. It can then be of advantage to re-filter the reaction mixture to obtain the pure glycerol carbonate.

The process according to the invention leads to glycerol carbonates with low percentage contents of free glycerol and polymeric glycerol carbonate. The glycerol carbonate thus obtained preferably contains at least 94% by weight and, more particularly, 95% by weight or more of glycerol carbonate.

The glycerol carbonate thus obtained is suitable for use in various fields of application, for example in washing aids and detergents. A particularly preferred application for high-purity glycerol carbonate is as a moisturizer in cosmetic preparations.

### EXAMPLES

100 g crude glycerol carbonate containing 5.4% by weight glycerol, 90.9% by weight glycerol carbonate and 3.7% by weight oligomeric glycerol carbonate according to HPLC analysis was reacted for 1 hour at 21° C. with the various bases listed in Table 1. After filtration of the reaction mixture, the pH was adjusted to 4-5 with phosphoric acid and the reaction mixture was re-filtered. The results of the reaction are also set out in Table 1 (results of HPLC analysis of the products).

TABLE 1

| | | | Glycerol | | |
|---|---|---|---|---|---|
| | | Quantity | carbonate | Glycerol | Oligomer |
| Batch | Base | % by wt. | % by wt. | % by wt. | % by wt. |
| A | — | — | 90.9 | 5.4 | 3.7 |
| B | NaOH conc. | 2 | 91.4 | 7.1 | 1.5 |
| C | Ca(OH₂) | 10 | 94.2 | 4.1 | 1.7 |
| D | Simet AP | 5 | 95.5 | 3.5 | 1.0 |
| E | Simet AP | 10 | 95.2 | 4.0 | 0.8 |
| F | Triethylamine | 5 | 95.4 | 4.0 | 0.6 |

Where bases C to F according to the invention are used, the glycerol carbonate is obtained in a higher purity than in known processes (batches A and B).

1-8 (cancelled).

9: A process for making a purified glycerol carbonate comprising:
(a) providing a mixture of crude glycerol carbonate, wherein said mixture contains glycerol carbonate, glycerol and oligomeric glycerol carbonate;
(b) providing a substantially water-free base component;
(c) reacting the mixture with the base component to form a reaction mixture; and
(d) filtering the reaction mixture to form the purified glycerol carbonate.

10: The process of claim 9 wherein the base component has a pK_{B} value of at most 5.

11: The process of claim 9 wherein the base component has a water content of at most about 5% by weight, based on the weight of the base component.

12: The process of claim 9 wherein step (c) is carried out at room temperature.

13: The process of claim 9 wherein the base is employed in an amount of from about 0.5 to 12% by weight, based on the weight of the crude glycerol carbonate.

14: The process of claim 9 wherein the base is employed in an amount of from about 1 to 10% by weight, based on the weight of the crude glycerol carbonate.

15: The process of claim 9 wherein the base is employed in an amount of from about 2 to 5% by weight, based on the weight of the crude glycerol carbonate.

16: The process of claim 9 further comprising adjusting the pH of the purified glycerol carbonate of step (d) to from about 4 to 5 to form a pH adjusted glycerol carbonate, and then re-filtering the pH adjusted glycerol carbonate to form a highly-purified glycerol carbonate.

17: The process of claim 16 wherein the highly-purified glycerol carbonate has a glycerol carbonate content of at least 94% by weight.

18: The process of claim 16 wherein the highly-purified glycerol carbonate has a glycerol carbonate content of about 95% by weight.

19: The product of the process of claim 9.

20: The product of the process of claim 10.

21: The product of the process of claim 11.

22: The product of the process of claim 12.

23: The product of the process of claim 13.

24: The product of the process of claim 14.

25: The product of the process of claim 15.

26: The product of the process of claim 16.

27: The product of the process of claim 17.

28: The product of the process of claim 18.
